(19) United States
(12) Patent Application Publication
(10) Pub. No.: US 2006/0257466 A1
(43) Pub. Date: Nov. 16, 2006

(54) Administration of macrophage targeted formulations of compounds which modulate cholesterol-metabolizing enzymes for treatment of atherosclerosis

(76) Inventors: Perry M. Kim, Inverary (CA); Robert Bender, Ottawa (CA)

Correspondence Address: Licata & Tyrrell P.C., 66 East Main Street, Marlton, NJ, 08053, US

(21) Appl. No.: 11/399,066

(22) Filed: Apr. 6, 2006

Related U.S. Application Data
(60) Provisional application No. 60/669,067, filed on Apr. 6, 2005.

(51) Int. Cl. A61K 39/395; A61K 31/56; A61K 31/455; A61K 31/401; A61K 31/366; A61K 31/22; A61K 9/127

(52) U.S. Cl. 424/450; 424/145.1; 977/907; 514/171; 514/356; 514/423; 514/460; 514/548

(57) ABSTRACT

Macrophage targeting formulations of compounds for treating atherosclerosis, in particular compounds which modulate cholesterol-metabolizing enzymes including, but not limited to acyl CoA:cholesterol acyl transferase (ACAT) inhibitors, cholesterol ester hydrolase (CEH) enhancers and combinations thereof, are provided. Methods for short term administration of these macrophage targeting formulations to promote regression and/or inhibit formation of atherosclerotic plaque, as well as to treat atherosclerosis, inflammation, coronary heart disease and cardiovascular disease are also provided.

This patent application claims the benefit of priority to U.S. Provisional Patent Application Ser. No. 60/669,067 filed Apr. 6, 2005, teachings of which are herein incorporated by reference in their entirety.

### FIELD OF THE INVENTION

Macrophage targeted formulations of compounds for treating atherosclerosis, in particular compounds which modulate cholesterol-metabolizing enzymes including, but not limited to, acyl CoA:cholesterol acyl transferase (ACAT) inhibitors, cholesterol ester hydrolase (CEH) enhancers and combinations thereof are provided. Short term administration of these macrophage targeted formulations of the present invention is useful in promoting regression and/or inhibiting formation of atherosclerotic plaque. Thus, short term administration of these macrophage targeted formulations of the present invention is useful in the treatment of atherosclerosis and inflammation, as well as coronary heart disease and cardiovascular disease.

### BACKGROUND OF THE INVENTION

Cardiovascular disease, including coronary heart disease caused by atherosclerosis, is the single largest killer of adults in North America (2002 Heart and Stroke Statistical Update). The development and progression of atherosclerosis in coronary arteries can lead to heart attacks and angina. In 1999 it was estimated that 12.6 million Americans had coronary heart disease. Approximately 1 in 5 deaths in 1999 were due to coronary heart disease, with a total US and Canadian mortality of over 500,000 and 42,000 individuals, respectively. It is estimated that over 102 million American adults have blood cholesterol levels that are either border-line high risk, or high risk of developing coronary heart disease. In addition to the immediate social and economic burden that heart attacks have on our health care system, there also is the considerable cost associated with the aftermath of a coronary heart disease event. About 25% of males and 38% of females will die one year after a heart attack, and death by coronary heart disease tends to occur during a person's peak productive years (BRFSS [1997], MMWR vol. 49, No. SS-2, Mar. 24, 2000, CDC/NCHS). There is also a further economic burden of coronary heart disease associated with premature and permanent disability of the labor force. In 1998, over $10 billion was paid to Medicare beneficiaries for coronary heart disease (Health Care Financing Review, Statistical Supplement [2000], HFCA).

Patients currently have a choice of a number of different drugs to treat cardiovascular disease/coronary heart disease. These drugs fall into various classes, including antihypertensives and antihyperlipidemics. Although these products have been shown to be beneficial in reducing the progression of coronary heart disease and preventing heart attacks, they can be limited in their effectiveness in some individuals because of low tolerability and, in some cases, mitigation of drug efficacy by the compensatory effects of the liver (Turley, S. D. Am. J. Managed Care 2002 8 (2 Suppl):S29-32).

Approximately 13 million North Americans are taking cholesterol-lowering drugs, and the majority of these individuals are now treated with the category of drugs known as statins. Cholesterol synthesis inhibitors (statins) are for the most part considered safe and highly effective. However, there have been some recent setbacks for this drug class. For example, the 2001 voluntary recall of Bayer's statin Baycol™, the delayed North American introduction of AstraZeneca's statin Crestor™, and the recent concerns about the health risks associated with long-term statin use (Clearfield, M. B., Expert Opin. Pharmacother. 2002 3:469-477) are indicative of the need for new drugs.

Thus, pharmaceutical companies are currently developing drugs that work via different mechanisms from that of the current marketed drugs. Treatment with two or more drugs that act through different mechanisms can, in fact, be additive or synergistic in their combined ability to reduce cholesterol levels (Brown, W. V. Am. J. Cardiol. 2001 87(5A): 23B-27B; Buckert, E. Cardiology 2002 97:59-66). Ezetimibe (Zetia , Merck), which was recently approved by the FDA, can significantly reduce cholesterol levels by itself. Furthermore, since Ezetimibe works by decreasing cholesterol absorption (i.e. blocks cholesterol transport), it can also be given with cholesterol synthesis inhibitors (statins) to decrease plasma cholesterol levels to a greater extent than when either drug is given alone (Davis et al. Arterioscler Thromb Vasc Biol. 2001 21:2031-2038; Rader, D. J. Am. J. Managed Care 2002 8 (2 Suppl): S40-44).

Companies such as Esperion Therapeutics, a division of Pfizer, are developing ways to increase the levels of HDL, the so-called “good cholesterol”, which plays a key role in the reverse cholesterol transport pathway, known to be important for the excretion of cholesterol out of the body.

Small molecule acyl CoA:cholesterol acyl transferase (ACAT) inhibitors such as avasimibe (Pfizer), eflucimibe (Eli Lilly), pactimibe (Sankyo), Sandoz 58-035 (Sandoz), SCH 48461, and F-1394 have been developed to decrease high plasma cholesterol levels, as a method of treating atherosclerosis. Studies in various animal species have shown that long-term administration (at least over several months) of small molecule ACAT inhibitors and other cholesterol absorption inhibitors, e.g. ezetimibe, leads to a significant reduction in plasma cholesterol levels (>50%) and a concurrent reduction in the formation or progression of atherosclerotic plaque (Delsing et al. Circulation 2001 103:1778-1786; Davis et al. Art. Thromb. Vasc. Biol. 2001 21:2032-2038).

Natural and synthetic peptides corresponding to the N-terminal region of serum amyloid A 2.1 protein also inhibit ACAT activity (Kisilevsky and Tam, Journal of Lipid Research 2003 44:2257-2269). These peptides can also promote in vitro and in vivo macrophage cholesterol export (Kisilevsky and Tam, Journal of Lipid Research 2003 44:2257-2269).

Administration of a liposomal formulation of a synthetic SAA peptide that enhances cholesterol ester hydrolase (CEH) activity and administration of a liposomal formulation of a synthetic SAA peptide that inhibits ACAT activity have been disclosed to prevent plaque development and/or regress existing plaque in mice. These liposomal formulations were administered to mice for 16 days or less (PCT Application No. PCT/CA2004/000846, filed Jun. 11, 2004 and published U.S. Application No. US2004/0265982A1, filed Jun. 10, 2004).

### SUMMARY OF THE INVENTION

The present invention provides macrophage targeted formulations of compounds which modulate cholesterol-metabolizing enzymes, and short term administration thereof to a subject to promote regression and/or inhibit further progression of an atherosclerotic lesion and/or inhibit formation of atherosclerotic plaque. Preferred compounds for use in these macrophage targeted formulations are small molecule compounds, preferably small molecule compounds with a molecular weight of 1500 daltons or less. Examples include but are not limited to, small molecule acyl CoA:cholesterol acyl transferase (ACAT) inhibitors, small molecule cholesterol ester hydrolase (CEH) enhancers and combinations thereof. By small molecule compounds it is meant to include small organic molecules as well as peptides, with the proviso that the peptide is not a fragment of serum amyloid A proteins, or a structural mimetic or variant thereof.

An aspect of the present invention relates to the use of these macrophage targeted formulations to modulate an activity of a cholesterol-metabolizing enzyme. In particular, the activity of ACAT and/or CEH can be modulated by short term administration of a macrophage targeted formulation of the present invention. In a preferred embodiment of the present invention, the enzymatic activity is modulated in vivo. More preferred is modulation of the enzymatic activity in humans.

Another aspect of the present invention relates to short term use of these macrophage targeted formulations to increase and/or promote the mobilization and efflux of stored cholesterol from macrophages located in atherosclerotic plaques. In a preferred embodiment of the present invention, the increase and/or promotion of the mobilization and efflux of stored cholesterol from macrophages located in atherosclerotic plaques occurs in humans.

Another aspect of the present invention relates to short term use of these macrophage targeted formulations to increase and/or promote the mobilization and efflux of stored cholesterol from macrophages located at sites of inflammation. In a preferred embodiment of the present invention, the increase and/or promotion of the mobilization and efflux of stored cholesterol from macrophages located at sites of inflammation occurs in humans.

Another aspect of the present invention relates to methods for treating and/or preventing atherosclerosis and/or regressing or decreasing formation of arterial atherosclerotic lesions in a subject comprising administering to a subject for a short term a macrophage targeted formulation of the present invention. In a preferred embodiment the subject is a human.

Another aspect of the present invention relates to methods for treatment of cardiovascular disease in a subject comprising administering to a subject for a short term a macrophage targeted formulation of the present invention. In a preferred embodiment the subject is a human.

Another aspect of the present invention relates to methods for treatment of coronary heart disease in a subject comprising administering to a subject for a short term a macrophage targeted formulation of the present invention. In a preferred embodiment the subject is a human.

Yet another aspect of the present invention relates to methods for treating or preventing inflammation in a subject comprising administering to a subject for a short term a macrophage targeted formulation of the present invention. In a preferred embodiment the subject is a human.

### BRIEF DESCRIPTION OF THE FIGURES

FIG. 1 is a line graph comparing in vivo macrophage cholesterol export in mice treated with a liposomal formulation of the ACAT inhibitor, Sandoz 58-035, at an estimated dose of 20 μg (open circles), mice treated with 20 μg of free non-liposome formulated Sandoz 58-035 (closed circles), and mice administered phosphate buffered saline (closed triangles).

### DETAILED DESCRIPTION OF THE FIGURES

The accumulation of cholesterol in vascular cells such as macrophages is a defining pathological feature of atherosclerosis. Macrophages are key cells in the storage and removal of lipids. When macrophages engulf significant amounts of cholesterol and other lipids, they are often referred to as foam cells (cholesterol-laden macrophages). The appearance of foam cells is an early and important pathological process in the formation and progression of an atherosclerotic plaque.

By the term “macrophage targeted” as used herein it is meant to include targeting to all macrophages, including, but not limited to, cholesterol-laden macrophages or foam cells as well as macrophages prior to their engulfing lipids.

Two enzymes are critical for maintaining cellular cholesterol balance, acyl CoA:cholesterol acyl transferase (ACAT) and cholesterol ester hydrolase (CEH). There are two forms of ACAT (i.e., ACAT-1 and ACAT-2). ACAT-1 is primarily located in the macrophage, while ACAT-2 is located in tissues such as the liver and intestine. Inhibition of ACAT-2 via long term administration (greater than 6 months) of an ACAT inhibitor has been shown to reduce cholesterol absorption and thus reduce plasma cholesterol levels. This reduction in plasma cholesterol levels also appears to be important in reducing the formation of atherosclerotic lesions (Delsing et al. Circulation 2001 103:1778-1786). However, long-term inhibition of ACAT, and in particular ACAT-2, in certain organs such as the adrenal glands is believed to be at least partially responsible for some of the toxicity seen with various small molecule ACAT inhibitors (Robertson et al. Tox. Sci. 2001 59:324-334).

Some small molecule ACAT inhibitors such as avasimibe (Pfizer) are capable of inhibiting both ACAT-1 and ACAT-2 activity and thus promote cholesterol export out of macrophages/foam cells as well as inhibit cholesterol absorption and reduce plasma cholesterol levels. Development and clinical testing of the ACAT inhibitors avasimibe and pactimibe were recently discontinued due to inefficacy (Tardiff et al. Circ. 2004 110:3372-3377; Nissen et al. New Eng. J. Med. 2006 354:1253-1263; Fazio et al. New Eng. J. Med. 2006 354:1307-1309).

Short-term administration (16 days) of an ACAT-inhibitory SAA peptide is effective in preventing the further development of atherosclerotic lesions in apoE knockout mice, without reducing plasma cholesterol levels (Kisilevsky et al. Journal of Lipid Research 2005 46:2091-2101).

Cholesterol ester hydrolase, also referred to as cholesterol esterase and cholesteryl ester hydrolase, promotes the removal or efflux of cholesterol from macrophages.

Co-administration of an ACAT inhibitory SAA peptide with a SAA peptide that enhances cholesterol ester hydrolase (CEH) activity results in the regression of atherosclerotic lesions (Kisilevsky et al. Journal of Lipid Research 2005 46:2091-2101). These results demonstrate that the prevention and/or regression of atherosclerotic lesions by natural or synthetic ACAT inhibitory SAA peptides are the result of a direct effect on ACAT within macrophages/foam cells residing within the atherosclerotic lesion and do not depend upon the reduction in cholesterol absorption and decrease in plasma cholesterol levels.

The inventors herein have now found that complexing of a small molecule compound which modulates a cholesterol-metabolizing enzyme with a macrophage targeting agent such as a lipid results in a formulation which is selectively effective, upon short term administration, at increasing and/or promoting the mobilization and efflux of stored cholesterol from macrophages located in atherosclerotic plaques. Such formulations are expected to exhibit fewer unwanted side effects including, but not limited to, decreased toxicity resulting from enzyme modulation in other organs and/or fewer drug-drug interactions. In a preferred embodiment, the small molecule compound of the macrophage targeted formulation comprises a small molecule ACAT inhibitor, preferably an ACAT-1 inhibitor or an ACAT-1/ACAT-2 inhibitor, a small molecule CEH enhancer, or a combination of a small molecule ACAT inhibitor and a small molecule CEH enhancer.

By “small molecule compound” as used herein it is meant to include small organic molecules as well as peptides which modulate a cholesterol-metabolizing enzyme, with the proviso that the peptide is not a fragment of serum amyloid A proteins, or a structural mimetic or variant thereof. In a preferred embodiment, the small molecule compound has a molecular weight of 1500 daltons or less, preferably 1000 daltons or less, more preferably 750 daltons or less. Examples of small molecule ACAT inhibitors useful in the present invention include, but are not limited to, Zetia™ (Merck), Avasimibe (Pfizer), Eflucimibe (Eli Lilly), Pactimibe (Sankyo), SCH 48461, and F-1394.

By “short term” as used herein, it is meant that the macrophage targeted formulation of the present invention is administered to a subject for a single continuous period of time of about one month or less, or intermittently, e.g., every other month, bimonthly, four times a year, or biyearly for continuous periods of one month or less. By “continuous period” or “continuous periods” of short term administration, it is meant to include, but is not limited to, daily administration, every other day administration, semiweekly administration, weekly administration, and biweekly administration, of a macrophage targeted formulation of the present invention.

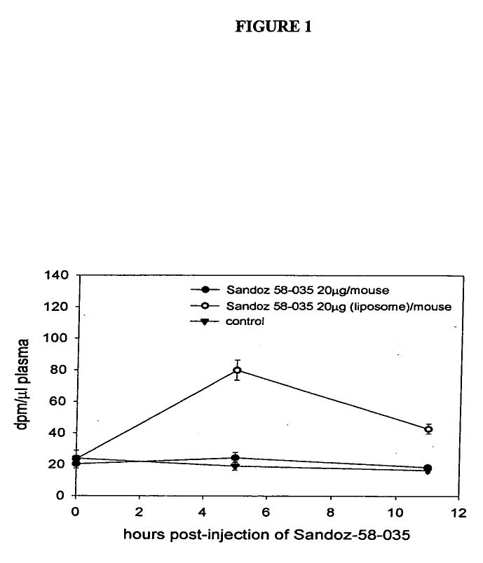

As shown in FIG. 1, a macrophage targeted formulation comprising a liposomal formulation of the ACAT inhibitor, Sandoz 58-035 at a dose estimated to be 20 μg or less was effective at enhancing in vivo macrophage cholesterol export (open circle plot) in mice. This enhancement was approximately 3-fold higher than that seen in mice treated with either 20 μg of free non-liposome formulated Sandoz 58-035 (closed circle plot) or PBS vehicle (closed triangle plot). In fact, at the dose administered, free non-liposome formulated Sandoz 58-035 did not increase macrophage cholesterol export over the vehicle (phosphate buffered saline) control group. Further, the dose of 20 μg of drug in the macrophage targeted formulation of the present invention administered in these experiments is believed to be an overestimate based upon 100% percent liposome incorporation of Sandoz 58-035. More realistically, percent liposome incorporation of Sandoz 58-035 is likely to be in the range of between 3% to 50%. Accordingly, enhanced efficacy of a macrophage targeted formulation as compared to an equal concentration of a free non-macrophage targeted formulation of the same drug at increasing macrophage cholesterol export is expected to be even greater than demonstrated by FIG. 1.

Since it is known that macrophages are the major route for in vivo liposome clearance, these data demonstrate for the first time that a macrophage targeted formulation such as this lipid complexed formulation increases the efficacy of small molecule compounds such as ACAT inhibitors which modulate cholesterol metabolizing enzymes. Further, efficacy of the macrophage targeted formulation was demonstrated within a short period of time and independently of plasma cholesterol reduction, indicating that short term administration (equal to or less than one month) of a single continuous period or intermittently for short term continuous periods will be effective in promoting regression and/or inhibiting formation of atherosclerotic plaque by directly acting on key cells within the lesion. Since the direct effects of small molecule compounds such as ACAT inhibitors on atherosclerotic plaque may require higher doses and plasma concentrations of the molecule than that required to reduce cholesterol absorption and decrease plasma cholesterol levels, shorter administration periods and targeted administration to macrophages via complexing the compound to a macrophage targeting agent such as a lipid is preferred, to reduce toxicity and/or unwanted side effects associated with these small molecule compounds, as well as drug-drug interactions associated with administration of these small molecule compounds. For example, in order for a non-macrophage targeted ACAT inhibitor to have similar efficacy to macrophage targeted formulations of the present invention, the dose administered and thus the effective plasma concentration of the non-macrophage targeted ACAT inhibitor within the individual would have to be increased. However, non-macrophage targeted formulations of small molecule compounds, such as small molecule ACAT inhibitors, readily distribute non-selectively throughout the body and into different tissues. In the case of small molecule ACAT inhibitors, this non-selective distribution can lead to toxicity or unwanted side effects and potentially result in drug-drug interactions. This was the case for avasimibe, wherein the drug-drug interactions were are not necessarily associated with inhibition of the ACAT enzyme within the target cells/tissue. Thus, the macrophage targeted formulations of the present invention provide a significant advantage in modifying the in vivo distribution and efficacy of small molecule compounds such as ACAT inhibitors, thereby limiting their deleterious effects, while at the same time increasing the efficacy at a given dose.

Preferred macrophage targeted formulations of the present invention comprise lipid complexed formulations, more preferably, encapsulation of the compound which modulates a cholesterol-metabolizing enzyme in a phospholipid vesicle. As demonstrated throughout the instant application, an exemplary phospholipid vesicle useful in the present invention is a liposome. The liposomal macrophage targeted formulations of the present invention comprising a small molecule compound which modulates a cholesterol-metabolizing enzyme can be prepared in accordance with any of the well known methods such as described by Epstein et al. (Proc. Natl. Acad. Sci. USA 1985 82:3688-3692), Hwang et al. (Proc. Natl. Acad. Sci. USA 1980 77:4030-4034), EP 52,322, EP 36,676; EP 88,046; EP 143,949; EP 142,641; Japanese Pat. Appl. 83-118008, and EP 102,324, as well as U.S. Pat. Nos. 4,485,045 and 4,544,545, the contents of which are hereby incorporated by reference in their entirety. In some embodiments, liposomes used in the present invention may be small (about 200-800 Angstroms). In other embodiment, larger liposomes may be preferred. The liposomes may be of a unilamellar type in which the lipid content is greater than about 10 mol. percent cholesterol, preferably in a range of 10 to 40 mol. percent cholesterol, the selected proportion being adjusted for optimal peptide therapy. In some embodiment, unilamellar type liposomes may be preferred given their uniformity. However, multilamellar liposomes can also be used. In some embodiments, liposomes without cholesterol may be preferred. Further, modified liposomes such as polysaccharide anchored liposomes (Sihorkar and Vyas (J. Pharm. and Pharmaceut. 2001 4:138-158) can be used.

In addition, as will be understood by those of skill in the art upon reading this disclosure, phospholipid vesicles other than liposomes can also be used.

Further, formulations of the present invention may comprise alternative macrophage targeting agents such as, but not limited to, macrophage targeting antibodies, ligands selective for macrophages, nanoparticle systems such as described by Chellat et al. (Biomaterials 2005 26:7260-7275), which are selectively engulfed by macrophages in a similar manner to liposomes, and erythrocytes, also selectively engulfed by macrophages (Magnani et al. Biotechnol. Appl. Biochem. 1998 28:1-6), which can encapsulate a small molecule compound in accordance with the present invention.

The macrophage targeted formulations of the present invention can be administered short term alone or in combination with another anti-atherosclerotic agent. For example, for macrophage targeted formulations of the present invention comprising a small molecule compound which enhances CEH, the macrophage targeted formulation can be administered to a subject in combination with an ACAT inhibitor. Exemplary ACAT inhibitors include but are not limited to Zetia™ (Merck), Avasimibe (Pfizer), Eflucimibe (Eli Lilly), Pactimibe (Sankyo), SCH 48461, and F-1394. Macrophage targeted formulations of the present invention may also be administered to a subject with an apolipoprotein-free cholesterol acceptor (Rothblat et al. J. Lipid Res. 1999 40:781-796; Li et al. Biochimica Biophysica Acta 1995 1259:227-234; Jian et al. J. Biol. Chem. 1998 273(10):5599-5606) such as, for example, cyclodextrin. Additional exemplary cholesterol-lowering drugs or agents which can be administered in combination with a macrophage targeted formulation of the present invention include, but are not limited to, statins, resins, bile acid sequestrants (Bays et al. Expert Opinion on Pharmacotherapy 2003 4(11):1901-38; Kajinami et al. Expert Opinion on Investigational Drugs 2001 11(6):831-5), niacin (Van et al. Am. J. Cardiol. 2002 89(11):1306-8; Ganji et al. J. Nutri. Biochem. 2003 14(6):298-305; Robinson et al. Progress in Cardiovasc. Nursing 2001 16(1):14-20; Knopp, R. H. Am. J. Cardiol. 2000 86(12A):51L-56L), liver X receptor agonists (Tontonoz et al. Molecular Endocrinology 2003 17:985-993), calcium (Ca2+) antagonists (Delsing et al. Cardiovasc. Pharmacol. 2003 42(1):63-70), modulators of peroxisome proliferator-activated receptors (PPARS; Lee et al. Endocrinology 2003 144:2201-2207), and inhibitors of cholesterol ester transfer protein (Le Goff et al. Pharmacol. Ther. 2004 101:17-38).

By “combination” as used herein it is meant to include administration of a single macrophage targeted formulation of the present invention which includes one or more small molecule compounds modulating a cholesterol-metabolizing enzyme and a second anti-atherosclerotic agent, as well as separate administration of a macrophage targeted formulation of the present invention comprising one or more small molecule compounds that modulates a cholesterol-metabolizing enzymes and a second anti-atherosclerotic agent simultaneously or within a selected period of time of one another.

Short term administration of macrophage targeted formulations of the present invention can also comprise administration via a coronary stent implanted into a patient. Coronary stents which elute a macrophage targeted formulation of the present invention can be prepared and implanted in accordance with well known techniques (See, for example, Woods et al. Annu. Rev. Med. 2004 55:169-78); al-Lamce et al. Med. Device Technol. 2003 14:12-141 Lewis et al. J. Long Term Eff. Med. Implants 2002 12:231-50; Tsuji et al. Int. J. Cardiovasc. Intervent. 2003 5:13-6).

Short term administration of the macrophage targeted formulations of the present invention is useful in modulating the activity of a cholesterol-metabolizing enzyme, and in particular, the activity of ACAT, more preferably ACAT-1 or ACAT-1/ACAT-2 and/or CEH. In a preferred embodiment, short term administration of the macrophage targeted formulations of the present invention is used to modulate enzymatic activity selectively in macrophages. More preferably, short term administration of the macrophage targeted formulations of the present invention is used to modulate enzymatic activity in vivo. More preferably, short term administration of the macrophage targeted formulations of the present invention is used to modulate enzymatic activity in mammals and in particular humans.

By the terms “modulate”, “modulation” and/or “modulating” as used herein it is meant any change, more particularly any increase or decrease in a cholesterol-metabolizing enzyme activity which promotes cholesterol efflux from macrophages. Thus, for ACAT, by “modulate” modulation” and/or “modulating” it is meant an inhibition or decrease in ACAT activity while for CEH it is meant an enhancement or increase in CEH activity.

Short term administration of the macrophage targeted formulations of the present invention is also useful in promoting the mobilization and efflux of stored cholesterol located in atherosclerotic plaques and/or sites of inflammation. In a preferred embodiment, short term administration of macrophage targeted formulations of the present invention is used to promote the mobilization and efflux of stored cholesterol from macrophages and other cells or tissues located in atherosclerotic plaques or sites of inflammation in mammals, and in particular humans.

Accordingly, the macrophage targeted formulations of the present invention can be administered short term to a subject, preferably a mammal, more preferably a human, to treat and/or prevent atherosclerosis and/or regress or decrease formation of arterial atherosclerotic lesions in a subject. The macrophage targeted formulations may be administered by various routes including, but not limited to, orally, intravenously, intramuscularly, intraperitoneally, topically, rectally, dermally, transdermally, subcutaneously, sublingually, buccally, intranasally, intraocularly or via inhalation. The route of administration as well as the dose and frequency of short term administration can be selected routinely by those skilled in the art based upon the severity of the condition being treated, as well as patient-specific factors such as age, weight and the like.

In addition to the above-described in vivo assays, efficacy of compositions of the present invention to treat and/or prevent atherosclerosis can also be demonstrated in an animal model such as the ApoE knockout mouse model of atherogenesis (Davis et al. Arterioscler Thromb Vasc Biol. 2001 21:2031-2038). These mice, when placed on an atherogenic diet, rapidly deposit lipid into their aortas. The ApoE knockout mice are a validated model of atherosclerosis and were used to demonstrate the effectiveness of Ezetimibe (Zetia™; Merck) in reducing atherosclerosis (Davis et al. Arterioscler Thromb Vasc Biol. 2001 21:2031-2038). The efficacy of short term administration of a liposomal formulation of the present invention in treating or preventing atherosclerosis can be demonstrated in similar fashion.

The in vivo effectiveness of short term administration of a macrophage targeted formulation of the present invention in preventing or reducing the degree of atherosclerosis, can be demonstrated in the above rodent model for atherogenesis. To demonstrate the ability of short term administration of a macrophage targeted formulation of the present invention to cause regression of atherosclerosis, the rodents are placed on an atherogenic diet such as described by Tam et al. (J. Lipid Res. 2005 46:2091-2101) for two weeks. The animals are then divided into three groups, one group which is sacrificed prior to treatment (used as a baseline for regression analysis), a second group which continues on the diet for at least an additional two weeks, and a third group which continues on the diet for the same period but also receives a macrophage targeted formulation of the present invention. The effects of this short term administration of a macrophage targeted formulation of the present invention on arterial atherosclerosis are assessed at the termination of the experiment, when the aortas are removed from the animals and opened longitudinally. The area of the endothelial surface occupied by lipid is measured via oil red O staining. Histological sections of aorta are also prepared for microscopic analysis and total lipids are isolated to measure the quantity of cholesterol (esterified or non-esterified) and triglycerides per wet weight of tissue.

These experiments in this well-accepted rodent model of atherosclerosis provide further evidence of short term administration of the macrophage targeted formulations of the present invention modulating cholesterol metabolic pathways in various tissues and/or cells. Using techniques such as pharmacokinetic scaling, these studies in rodents can be used to predict disposition and define pharmacokinetic equivalence and to design dosage regimens in other species including humans (Mordenti, J. J. Pharmaceutical Sciences 1986 75(11):1028-1040). By dosing regimens it is meant to include the amount of drug administered, the intervals at which the drug is administered during a continuous short term period of administration, as well as the number of intermittent short term administration periods and the length of time of the intervals between these intermittent short term administration periods.

Administration of pharmaceutical compositions of the present invention is also expected to be useful in the treatment of coronary heart disease and cardiovascular disease and in the prevention or treatment of inflammation.

The invention is further illustrated by the following examples, which should not be construed as further limiting. The contents of all references, pending patent applications, and published patents cited throughout this application are hereby expressly incorporated by reference.

### EXAMPLES

### Example 1

### Animals

Swiss-white CD1 6-8 week old female mice were obtained from Charles River, Montreal, Quebec. Mice were kept in a temperature controlled room on a 12 hour light/dark cycle. They were fed with Purina Lab Chow pellets and water ad libitum.

### Example 2

### Chemicals

All chemicals were reagent grade and purchased from Fisher Scientific (Nepean, Ont.), Sigma (St. Louis, Mo.), ICN (Aurora, Ohio), or BioRad (Hercules, Calif.). Dulbecco's Modified Eagle's Medium (DMEM) and fetal bovine serum (FBS) were purchased from Life Technologies (Burlington, Ont.). Radiolabeled [1,2,6,7-³H(N)]-cholesterol (82 Ci/mmol) was obtained from Perkin Elmer.

### Example 3

### ACAT Inhibitor Formulation

A stock solution of Sandoz 58-035 (Sandoz stock solution) was prepared by dissolving Sandoz 58-035 in dimethyl sulphoxide (Sigma cat.# D-2650) to a final concentration of 2 mg/ml.

For non-liposome formulated Sandoz 58-035 experiments, 10 ul of the stock solution (2 mg/ml) was diluted with 190 ul of phosphate buffered saline (PBS) to give a final Sandoz 58-035 solution of 20 μg/200 μl. Two hundred microliters of this solution containing 20 μg of Sandoz 58-035 was injected into each mouse through the tail vein.

Sandoz 58-035 was liposome formulated using a modified method of Jonas and co-workers (Jonas et al. J. Bio. Chem. 1989 264:4818-4825). For this formulation, a Sandoz 58-035 solution was prepared by taking 0.5 ml of the Sandoz stock solution (2 mg/ml, see above) and diluting it with 9.5 ml of PBS, containing 53.75 mg cholic acid. Separately, phospholipid (33.9 mg) and cholesterol (4.83 mg) were dissolved in chloroform and dried under a stream of nitrogen forming a thin film. To prepare a volume of 10 ml of Sandoz 58-035 liposomes, this thin film of dried lipid was hydrated with the solution of Sandoz 58-035 in PBS containing cholic acid (53.75 mg). The resulting mixture of lipid and 1 mg of Sandoz 58-035 was vortexed overnight at 4° C. to form the liposome containing Sandoz 58-035 formulation. The newly formed liposome formulation was extensively dialyzed with 1 liter of PBS, which was changed four times. This dialysis procedure removed the cholic acid and any free non-liposome formulated Sandoz 58-035 compound. Two hundred microliters of the liposome formulation was injected into the tail vein of each mouse and cholesterol export measurements were taken as described in below (see Example 5).

### Example 4

### Preparation of Red Blood Cell Membranes as a Source of Cholesterol

To mimic the ingestion of cell membrane fragments by macrophages at sites of tissue injury, red blood cell membrane fragments were prepared and used as a source of cholesterol in accordance with the procedure described by Ely et al. (Amyloid 2001 8:169-181). Similar quantities of cholesterol (as red blood cell membrane fragments) were used in all experiments. The concentration of cholesterol in the red blood cell membrane preparations was determined using the method of Allain and co-workers (Clin. Chem. 1974 20:470-475), with the aid of a Sigma cholesterol 20 reagent kit (Sigma Chemical Co., St. Louis, Mo.).

### Example 5

### Cell Culture

J774 macrophages (from American Type Culture Collection, Manassas, Va.; ATCC #T1B-67) were maintained at 1 million cells per well and grown in 2 mL of DMEM supplemented with 10% FBS to 90% confluence. The medium was changed 3 times a week.

### Example 6

### Cholesterol Efflux in vivo

To determine cholesterol export in vivo, J774 cells were loaded with cholesterol and simultaneously incubated for 3 hours with 0.5 μCi/mL [³H]-cholesterol, followed by an overnight equilibration period as described by Tam et al. (J. Lipid Res. 2002 43:1410-1420). Cells were washed four times with PBS/BSA prior to the efflux studies and then detached from the culture dishes. Five million cells in 200 μl DMEM were injected into control mice or inflamed mice through the tail vein. Cells were allowed to establish within the mice for 24 hours. At 24 hours, mice (n=4/group) were treated with either 20 μg of Sandoz 58-035 (PBS diluted stock solution), approximately 20 μg of Sandoz 58-035 in a liposome formulation or phosphate buffered saline. At various time points, approximately 25 μl of blood were collected from the tail vein of each animal into heparinized capillary tubes and then centrifuged for 5 minutes in an Adams Autocrit Centrifuge to separate red blood cells from plasma. Cholesterol efflux was determined by measuring the appearance of [³H]-cholesterol in plasma by scintillation spectrometry. Results are depicted in FIG. 1.

What is claimed is:

1. A method for increasing or promoting mobilization and efflux of stored cholesterol from macrophages located in atherosclerotic plaques in a subject comprising administering to a subject for a short term a macrophage targeted formulation comprising a small molecule compound which modulates a cholesterol metabolizing enzyme, with the proviso that the small molecule compound is not a peptide fragment of a serum amyloid A protein, or a structural mimetic or variant thereof, and a macrophage targeting agent.

2. The method of claim 1 wherein the small molecule compound is an acyl CoA:cholesterol acyl transferase (ACAT) inhibitor and the activity of the cholesterol-metabolizing enzyme ACAT is inhibited.

3. The method of claim 1 wherein the small molecule compound is a cholesterol ester hydrolase (CEH) enhancer and the activity of the cholesterol-metabolizing enzyme CEH is enhanced.

4. The method of claim 1 wherein the macrophage targeted formulation comprises an ACAT inhibitor and a CEH enhancer.

5. The method of claim 1 wherein the macrophage targeting agent is selected from the group consisting of a lipid, a macrophage targeting antibody, a macrophage targeting ligand, a nanoparticle and an erythrocyte.

6. The method of claim 1 wherein the macrophage targeting agent is a phospholipid which encapsulates the small molecule compound.

7. The method of claim 1 wherein the macrophage targeting agent is a liposome.

8. The method of claim 1 wherein the macrophage targeting agent is a modified liposome.

9. The method of claim 1 further comprising administering a second anti-atherosclerotic agent.

10. The method of claim 9 wherein the second anti-atherosclerotic agent is an apolipoprotein free cholesterol acceptor, a statin, a resin, a bile acid sequestrant, niacin, a liver X receptor agonist, a calcium antagonist or a modulator of peroxisome proliferator-activated receptors.

11. The method of claim 1 wherein the subject is a human.

12. A method for increasing or promoting mobilization and efflux of stored cholesterol from macrophages located at sites of inflammation in a subject comprising administering to a subject for a short term a macrophage targeted formulation comprising a small molecule compound which modulates a cholesterol metabolizing enzyme, with the proviso that the small molecule compound is not a peptide fragment of a serum amyloid A protein, or a structural mimetic or variant thereof, and a macrophage targeting agent.

13. The method of claim 12 wherein the small molecule compound is an acyl CoA:cholesterol acyl transferase (ACAT) inhibitor and the activity of the cholesterol-metabolizing enzyme ACAT is inhibited.

14. The method of claim 12 wherein the small molecule compound is a cholesterol ester hydrolase (CEH) enhancer and the activity of the cholesterol-metabolizing enzyme CEH is enhanced.

15. The method of claim 12 wherein the macrophage targeted formulation comprises an ACAT inhibitor and a CEH enhancer.

16. The method of claim 12 wherein the macrophage targeting agent is selected from the group consisting of a lipid, a macrophage targeting antibody, a macrophage targeting ligand, a nanoparticle and an erythrocyte.

17. The method of claim 12 wherein the macrophage targeting agent is a phospholipid which encapsulates the small molecule compound.

18. The method of claim 12 wherein the macrophage targeting agent is a liposome.

19. The method of claim 12 wherein the macrophage targeting agent is a modified liposome.

20. The method of claim 12 further comprising administering a second anti-atherosclerotic agent.

21. The method of claim 20 wherein the second anti-atherosclerotic agent is an apolipoprotein free cholesterol acceptor, a statin, a resin, a bile acid sequestrant, niacin, a liver X receptor agonist, a calcium antagonist or a modulator of peroxisome proliferator-activated receptors.

22. The method of claim 12 wherein the subject is a human.

23. A method for treating or preventing atherosclerosis or regressing or decreasing formation of arterial atherosclerotic lesions in a subject comprising administering to a subject for a short term a macrophage targeted formulation comprising a small molecule compound which modulates a cholesterol metabolizing enzyme, with the proviso that the small molecule compound is not a peptide fragment of a serum amyloid A protein, or a structural mimetic or variant thereof, and a macrophage targeting agent.

24. The method of claim 23 wherein the small molecule compound is an acyl CoA:cholesterol acyl transferase (ACAT) inhibitor and the activity of the cholesterol-metabolizing enzyme ACAT is inhibited.

25. The method of claim 23 wherein the small molecule compound is a cholesterol ester hydrolase (CEH) enhancer and the activity of the cholesterol-metabolizing enzyme CEH is enhanced.

26. The method of claim 23 wherein the macrophage targeted formulation comprises an ACAT inhibitor and a CEH enhancer.

27. The method of claim 23 wherein the macrophage targeting agent is selected from the group consisting of a lipid, a macrophage targeting antibody, a macrophage targeting ligand, a nanoparticle and an erythrocyte.

28. The method of claim 23 wherein the macrophage targeting agent is a phospholipid which encapsulates the small molecule compound.

29. The method of claim 23 wherein the macrophage targeting agent is a liposome.

30. The method of claim 23 wherein the macrophage targeting agent is a modified liposome.

31. The method of claim 23 further comprising administering a second anti-atherosclerotic agent.

32. The method of claim 31 wherein the second anti-atherosclerotic agent is an apolipoprotein free cholesterol acceptor, a statin, a resin, a bile acid sequestrant, niacin, a liver X receptor agonist, a calcium antagonist or a modulator of peroxisome proliferator-activated receptors.

33. The method of claim 23 wherein the subject is a human.

34. A method for modulating an activity of a cholesterol-metabolizing enzyme in a subject comprising administering to a subject for a short term a macrophage targeted formulation comprising a small molecule compound which modulates a cholesterol metabolizing enzyme, with the proviso that the small molecule compound is not a peptide fragment of a serum amyloid A protein, or a structural mimetic or variant thereof, and a macrophage targeting agent.

35. The method of claim 34 wherein the small molecule compound is an acyl CoA:cholesterol acyl transferase (ACAT) inhibitor and the activity of the cholesterol-metabolizing enzyme ACAT is inhibited.

36. The method of claim 34 wherein the small molecule compound is a cholesterol ester hydrolase (CEH) enhancer and the activity of the cholesterol-metabolizing enzyme CEH is enhanced.

37. The method of claim 34 wherein the macrophage targeted formulation comprises an ACAT inhibitor and a CEH enhancer.

38. The method of claim 34 wherein the macrophage targeting agent is selected from the group consisting of a lipid, a macrophage targeting antibody, a macrophage targeting ligand, a nanoparticle and an erythrocyte.

39. The method of claim 34 wherein the macrophage targeting agent is a phospholipid which encapsulates the small molecule compound.

40. The method of claim 34 wherein the macrophage targeting agent is a liposome.

41. The method of claim 34 wherein the macrophage targeting agent is a modified liposome.

42. The method of claim 34 further comprising administering a second anti-atherosclerotic agent.

43. The method of claim 42 wherein the second anti-atherosclerotic agent is an apolipoprotein free cholesterol acceptor, a statin, a resin, a bile acid sequestrant, niacin, a liver X receptor agonist, a calcium antagonist or a modulator of peroxisome proliferator-activated receptors.

44. The method of claim 34 wherein the subject is a human.

45. A method for treatment of cardiovascular disease, coronary heart disease, or inflammation in a subject comprising administering to a subject for a short term a macrophage targeted formulation comprising a small molecule compound which modulates a cholesterol metabolizing enzyme, with the proviso that the small molecule compound is not a peptide fragment of a serum amyloid A protein, or a structural mimetic or variant thereof, and a macrophage targeting agent.

46. The method of claim 45 wherein the small molecule compound is an acyl CoA:cholesterol acyl transferase (ACAT) inhibitor and the activity of the cholesterol-metabolizing enzyme ACAT is inhibited.

47. The method of claim 45 wherein the small molecule compound is a cholesterol ester hydrolase (CEH) enhancer and the activity of the cholesterol-metabolizing enzyme CEH is enhanced.

48. The method of claim 45 wherein the macrophage targeted formulation comprises an ACAT inhibitor and a CEH enhancer.

49. The method of claim 45 wherein the macrophage targeting agent is selected from the group consisting of a lipid, a macrophage targeting antibody, a macrophage targeting ligand, a nanoparticle and an erythrocyte.

50. The method of claim 45 wherein the macrophage targeting agent is a phospholipid which encapsulates the small molecule compound.

51. The method of claim 45 wherein the macrophage targeting agent is a liposome.

52. The method of claim 45 wherein the macrophage targeting agent is a modified liposome.

53. The method of claim 45 further comprising administering a second anti-atherosclerotic agent.

54. The method of claim 53 wherein the second anti-atherosclerotic agent is an apolipoprotein free cholesterol acceptor, a statin, a resin, a bile acid sequestrant, niacin, a liver X receptor agonist, a calcium antagonist or a modulator of peroxisome proliferator-activated receptors.

55. The method of claim 45 wherein the subject is a human.
